(19) United States
(12) Patent Application Publication
(10) Pub. No.: US 2004/0086499 A1
(43) Pub. Date: May 6, 2004

(54) Method of immobilising molecules and particles on a hydrophobic polymer surface wherein mucin is used

(76) Inventors: Karin Dahlgren Caldwell, Djursholm (SE); Tomas Sandberg, Uppsala (SE); Lei Shi, San Antonio, TX (US); Maria Werner, Uppsala (SE); Matti J. Nikkola, Sundbyberg (SE)

Correspondence Address: AMERSHAM BIOSCIENCES, PATENT DEPARTMENT, 800 CENTENNIAL AVENUE, PISCATAWAY, NJ, 08855, US

(21) Appl. No.: 10/432,515

(22) PCT Filed: Dec. 7, 2001
(86) PCT No.: PCT/SE01/02711

(51) Int. Cl. A61F 13/00; A61K 38/43

(52) U.S. Cl. 424/94.1; 424/422

(57) ABSTRACT

The present invention generally relates to a method of immobilising molecules or particles on the surface of hydrophobic polymeric materials, wherein said immobilisation is mediated by a mucin layer adsorbed to said surface. More particularly the invention relates to such a method, wherein the polymeric material is a biomaterial for in vivo applications. The present invention also relates to a hydrophobic polymeric substrate provided with a mucin layer having selected molecules and/or particles immobilised thereto, obtainable by means of the method, and a biocompatible highly specific binding system comprising a mucin and a lectin or a functional fragment of a lectin, which system can be used in the method.

### FIELD OF THE INVENTION

The present invention generally relates to a method of immobilising molecules or particles on the surface of hydrophobic polymeric materials, wherein said immobilisation is mediated by a mucin layer formed onto said surface. In one particular embodiment the invention relates to such a method, wherein the polymeric material is a biomaterial for in vivo applications.

### TECHNICAL BACKGROUND

In today's surgical procedures, polymeric materials are commonly implanted into the human body for temporary or permanent use. A limiting factor for the use of these biomaterials is the risk of infection associated with microbial colonisation. Staphylococcus aureus and the coagulase negative staphylococcus (CNS) S. epidermidis are the most frequently encountered biomaterial-associated pathogens. Another limiting problem is that of rejection and encapsulation (foreign body effects), which are frequently encountered during in vivo use of known coated substrates, especially in long term in vivo systems

It is generally believed that depending on the degree of hydrophobic interaction, microbes will more or less easily adhere to artificial surfaces, where they usually proliferate extensively. A higher degree of interaction would thus correspond to an increased probability of adherence.

Lei Shi et al., in Mucin coating on polymeric material surfaces to suppress bacterial adhesion, Colloids and Surfaces B: Biointerfaces 17 (2000), 229-239, describe coating of bovine submaxillary gland mucin (BSM) onto surfaces of different polymeric materials, such as PMMA, silicone, Tecoflex® (a medical grade polyurethane) and polystyrene. Suppression of bacterial adhesion by Staphylococcus aureus and CNS Staphylococcus epidermidis on the BSM coated surfaces of said materials is reported. A correlation between the suppression and the surface concentration of adsorbed mucin is also said to be observed, viz. the more mucin that is coated on these surfaces, the less bacteria will adhere to them. A significantly reduced surface hydrophobicity after mucin coating is also observed. It is suggested that mucin coatings could profitably be employed to reduce the risk of microbial infections on polymeric biomaterials.

U.S. Pat. No. 5,516,703 pertains to the field of biological separation, such as low pressure affinity chromatography and immunological assays, and the problems encountered with hydrophobic surfaces. The specification states that hydrophobic surfaces, such as those of polystyrene, are nonspecifically active to the adsorption of various substances, such as biomolecules with hydrophobic portions, proteins and the like. Attempts in the prior art to form specifically active surfaces on such nonspecifically active surfaces include covalent bonding to the surface of ligands with specific activity, and simple adsorption of biological molecules such as enzymes and antibodies onto the solid surface. The adsorbed biomolecule will then provide a specific enzymatic reaction or specific antibody-antigen reaction. These approaches are both said to be associated with problems. Accordingly, a covalent coating, as a rule, is hard to remove from the substrate and may not produce complete coverage, thus allowing uncovered areas to engage in undesirable nonspecific adsorption of protein. Some biological molecules are difficult to adsorb and some enzymes and antibodies lose activity when adsorbed upon a hydrophobic surface. Thus, the major objects of the invention is to provide a coating for a hydrophobic substrate that provides the surface with specifically reactive sites at a predetermined concentration, and to provide a hydrophilic protein compatible coating for hydrophobic substrates with little or no background nonspecific reactivity. The objects are achieved by means of coating hydrophobic surfaces with a PEO-PPO-PEO tri-block copolymer, i.e. polymers of the Pluronic® type, in order to render the surfaces protein resistant while permitting covalent attachment of specific ligands. According to said invention the ends of block surfactant polymers with hydrophilic pendant blocks attached to a hydrophobic block are reacted to form a derivative of the surfactant polymer with specifically active sites at the free ends of the hydrophilic blocks. The derivative is then adsorbed onto the hydrophobic substrate to produce a surface with a minimum of nonspecific activity from the hydrophobic substrate and with specific activity provided by the block copolymer derivative.

However, in many biomedical applications, such as the above-mentioned, it would be desirable to have a method available, by means of which molecules or particles could be immobilised on a hydrophobic biopolymer surface intended for use in an in vivo application. An essential requirement in this case is that such method must offer a sufficiently high level of biocompatibility. In for example the case of implants, it would be desirable to be able to immobilise certain molecules or particles on the surface of the implant, such as for example for the purpose of sustained release of a pharmaceutical component from the surface of an implant, or for the directional endothelisation. As already mentioned, such implants are often made of polymeric biomaterials, and exhibit a hydrophobic surface due to the nature of the polymeric material, and are therefore liable to microbial colonisation. Accordingly, the risk of microbial infection must also be minimised by such method. Accordingly, it is an object of the present invention to provide a method by means of which selected molecules and particles can be immobilised on the surface of a hydrophobic polymeric biomaterial with improved biocompatibility, while minimising the risk of microbial infections.

Such method is provided according to claim 1 of the present invention, wherein selected molecules and/or particles are immobilised on a surface of a hydrophobic polymeric material by means of the following steps:

forming a mucin layer onto a surface of a substrate of a hydrophobic polymeric material; and

adsorbing and/or covalently attaching said molecules and/or particles to the mucin layer formed.

Other embodiments and advantages will be evident from the following description and dependent claims.

According to another aspect, the present invention is directed to a hydrophobic polymeric substrate provided with a mucin layer having selected molecules and/or particles immobilised thereto, obtainable by means of the inventive method.

According to a further aspect, the present invention is directed to a biocompatible highly specific binding system comprising a mucin and a lectin or a functional fragment of a lectin, which system can be used in the inventive method.

### SUMMARY OF THE PRESENT INVENTION

According to the present invention it has unexpectedly been found that a layer of mucin formed on a hydrophobic surface of a polymer substrate, will provide an excellent binding matrix to the surface, while also markedly improving the biocompatibility of the substrate surface as compared to a the prior art coatings.

Accordingly, the present inventors have found a mucin coating to be recognised by an animal body as being close to endogenous, or even essentially endogenous, and thus, that the resulting mucin surface coating of the present invention will markedly alleviate the problems of rejection and encapsulation frequently encountered during use of previously known coated substrates in vivo, especially in long term in vivo systems.

According to the present invention it has been found that molecules or particles can be immobilised with a very high degree of specificity on a layer of mucin formed on a hydrophobic surface. At the same time, the use of mucin according to the present invention has also been found to significantly reduce the nonspecific binding to the coated hydrophobic surface, as compared to the prior art coating systems, such as, for example, PEO-PPO-PEO tri-block copolymers. The reason for this is likely to be due to a combination of a lower residual nonspecific binding activity of the hydrophobic surface after formation of a mucin layer thereon, and also to that the exposed resulting mucin layer on the hydrophobic surface, exhibits a very low nonspecific adsorption.

In one embodiment of the method of the present invention the mucin layer is formed by covalent attachment to the hydrophobic surface, such as by amination of the substrate surface.

In an alternative embodiment, the mucin layer is formed by adsorption to the substrate surface.

According to the method of the invention, immobilisation molecules and/or particles can be achieved by means of adsorption, i.e. non-covalent binding to the matrix due to affinity, or by means of covalent binding to activated exposed groups of the mucin molecules, or a combination thereof. This will be described in more detail below.

The present inventors have found a certain group of molecules which binds to the exposed mucin surface with a very high degree of specificity. Accordingly, in another aspect, the present invention relates to a biocompatible highly specific binding system for immobilising particles or molecules, which system comprises a mucin and a lectin.

The combination of a resulting very low nonspecific binding to the mucin layer and the possibility of binding specific molecules or particles to the mucin matrix makes the mucin coating an excellent binding matrix for carefully regulated specific binding of desired molecules or particles in both in vivo and ex vivo applications, where a very low nonspecific adsorption of macromolecules and particles is an important requirement.

Examples of such in vivo and ex vivo applications are the binding/association of cell-adhesion molecules such as fibronectin, laminin, or similar integrin receptors, including peptide sequences containing the RGD adhesion motif as well as the binding of specific growth factors.

The nonspecific binding can be further reduced when purified and, preferably, highly purified mucin is used. Accordingly, in a further embodiment, the method of the present invention also includes a purification step. The purification is primarily intended to remove low molecular weight species from the mucin.

Thus, purification of the mucin is especially preferred in cases where it is important that nonspecific binding is reduced or kept low. This is for example generally the case for an in vivo application.

Furthermore, the mucin coating is biodegradable, which in many in vivo applications is desirable as well as in model studies in vitro.

As will readily be recognised by the person skilled in the art, a high degree of biocompatibility is also highly desirable in providing a model surface for protein and cell studies on a hydrophobic substrate.

As also will be recognised by the person skilled in the art, these properties, together with a low non-specific binding, are also of great importance in analytical methods, such as in multi array technology, in biosensor applications, or in applications within the field of mass spectrometry with the mucin matrix acting as a capturing matrix, especially for proteins or fragments thereof.

As used herein, the term hydrophobic is intended to denote a surface having a water contact angle of greater than about 60°, preferably greater than about 70°. Examples of such materials are polystyrene (PS), polypropylene (PP), polyethylene (PE), polyvinylchloride (PVC), silicone rubbers, polycarbonates (PC), polyethylene terephtalate (PET), polymethyl methacrylate (PMMA), polytetrafluoroethylene (PTFE) and aminated polytetrafluoroethylene (NH-PTFE).

### BRIEF DESCRIPTION OF THE ATTACHED DRAWINGS

FIG. 1 shows polyacrylamide gel electrophoresis (PAGE) on crude and purified BSM under native (A and B) and SDS denaturing (C and D) conditions. Gels are stained for proteins (A) and proteins/carbohydrates (B-D). In the figure A represents silver protein staining, B silver-PAS protein/carbohydrate staining, C denaturing and non-reduced conditions, and D denaturing and reduced (2-mercapto ethanol) conditions.

BSM: Crude BSM, QS1A: Purified BSM (BSM-1), HMW/LMW: Molecular weight standards

FIG. 2 shows Western blot of PAGE 8-25 using an anti-BSA antibody system. A. Western blot of native PAGE using gradient 8-25 gel and an anti-BSA antibody system. Lane 1=crude BSM, 0.5 mg/ml; lane 2=crude BSM 0.25 mg/ml; lane 3=purified BSM (BSM-2), 0.5 mg/ml; lane 4=purified BSM, 0.25 mg/ml; lane 5=bovine serum albumin (BSA), 0.25 mg/ml and lane 6=High molecular weight standard. B. Whole sample analysis using anti-BSA antibody system. Numbers indicate concentration in μg/ml of each component. Samples were analyzed in duplicates.

FIG. 3 shows the water contact angles for non-coated and mucin-coated (before and after wash at shear rate 50 s⁻¹ for 1 hour) substrates coated by adhesion over night.

FIG. 4 shows the water contact angles for non-coated and mucin-coated substrates coated by drying at 37° C.

FIG. 5 shows the protein uptake of HSA, IgG, fibronectin and lysozyme by differently coated polystyrene (PS) surfaces.

FIG. 6 shows the adhesion of Pseudomonas aeruginosa (laboratory strain CCUG) to mucin-coated and uncoated polystyrene surfaces after different wash times (shear rate 29 s⁻¹). At right are microscopic views of uncoated and mucin-coated polystyrene surfaces.

FIG. 7 shows the adhesion of Pseudomonas aeruginosa (clinical strains) to mucin-coated and uncoated polystyrene surfaces after 60 minutes wash (shear rate 29 s¹).

In FIG. 8 a comparison of Pseudomonas aeruginosa adhesion to PS surfaces coated with different mucin subtypes is illustrated.

FIG. 9 represents microscopic views (2.5×) showing purified BSM and Pluronic F108 drop-coated surfaces exposed to human fibronectin. Results of a subsequent 24 hours incubation with human peripheral neuronal blastoma cells (SH 45 Y) are depicted.

FIG. 10 shows the relative binding of SA-HRP to differently coated polystyrene surfaces after subsequent 1 hour incubation with biotinylated jacalin (B-J)/streptavidin-HRP (SA-HRP) conjugate or TBS/SA-HRP control. TBS and bare polystyrene served as negative controls.

FIG. 11 shows a diagram over the relative IgG binding to differently treated NH-PTFE surfaces.

FIG. 12 shows absorbance readings of developed microtiterplate wells coated with mucin and IgA at two different concentrations, followed by screening for jacalin binding using anti-His-HRP antibody in combination with ABTS substrate.

FIG. 13 shows microscopic views at 40× magnification of histological stains of PU implant pre-coated with purified BSM, Pluronic F108 and Fibrinogen, respectively.

### DETAILED DESCRIPTION OF THE PRESENT INVENTION

Mucin, representing a group of large glycoproteins, constitutes one of the major components of mucus, which covers the lumenal surfaces of epithelial organs and serves as a physical barrier between the extracellular milieu and the plasma membrane. The molecules have a generic structure consisting of a thread-like peptide backbone with alternating regions decorated of densely packed carbohydrate side chains. Protein and carbohydrate contents are about 20-60 and 40-80%, respectively.

According to one embodiment of the method of the present invention, nonspecific adsorption characteristics exhibited by hydrophobic surfaces are utilised to adsorb the mucin molecules. When mucin is brought into contact with a hydrophobic material in aqueous environment, the naked parts of mucin's protein backbone will adhere due to its hydrophobicity, while the hydrophilic carbohydrate side chains are thought to orient themselves away from the surface.

The mucins are somewhat unique in that their carbohydrate moieties consist to a large extent of O-linked oligosaccharides, which attach to the peptide backbone via its serine and threonine residues. The number of sugar residues per oligosaccharide side chain varies from 1-20; their composition is mainly of the GalNAc, GlcNAc, galactose, fucose and sialic acid type. Immobilisation of molecules and particles to the mucin layer according to the present invention is thought to be accomplished by adsorption, i.e. due to either affinity to any of the groups, or to a certain sequence or combination of groups, contained in an oligosaccharide side chain, or by covalent bonding to an activated group of such oligosaccharide side chains.

Mucins are for example found at the epithelial cell lining of animals and in certain animal secretions, e.g. saliva and tears, a specific example being bovine submaxillary gland mucin (BSM)

According to one embodiment of the method of immobilisation of the present invention, commercially available mucin is used. To the resulting mucin matrix, any molecules having affinity to exposed moieties of the mucin molecule can be immobilised by adsorption. A suitable example of such molecules are the lectins, as will be described in more detail below. A desired molecule or particle can be attached or linked to a molecule having affinity for the exposed moieties of the mucin, such as a lectin molecule, by known linking or coupling methods, optionally including additional linking molecules. Such desired molecule or particle can then be immobilised to the mucin matrix by adsorption. Accordingly, particles and molecules which would otherwise not be adsorbed on a mucin layer can be linked to a molecule having the required affinity to mucin for adsorption to take place. Thus, hydrophobic particles and molecules (which would be nonspecifically adsorbed to a hydrophobic surface) can be adsorbed with a high specificity to the hydrophilic mucin layer by means of the specificity offered by for example a lectin molecule linked to such particle or molecule.

Immobilisation to the mucin matrix can also be covalent. Generally, in such case, the mucin matrix is activated in order to provide the desired reactivity. Molecules or particles exhibiting a desired matching functionality can then be immobilised to the matrix by reacting with the activated functionality of the mucin molecule. Such activation of the mucin matrix can for example be through oxidation of the sialic acid group, such as end terminal sialic acid groups, as well as other carbohydrates in the core structure. As an example, mild oxidation of sialic acid, and to a lesser degree oxidation of other carbohydrates with periodic acid (1 mM) leads to aldehyde functionality. Existing coupling chemistry for aldehydes can then be used for attachment of any macromolecule or particle (targets) containing one of the following functional groups: an amine group (amide formation by reductive amination using cyanoborohydride, as is known in the art); a hydrazide group (hydrazone formation stabilised by reducing agents).

For use with targets lacking one of the above-mentioned functional groups, a reagent, such as for example a heterobifunctional reagent, can be used for activation of other functionalities exhibited by the target molecule or particle. Accordingly, as an example, sulfhydryl groups or other groups, preferably occurring on specific sites on the molecule or particle, could be activated. Examples of suitable reagents are, for amine activation of sulfhydryl groups: Mercaptoalkylamine; and for hydrazide activation of sulfhydryl groups: 4-(4-N-maleimidophenyl) butyric acid hydrazide (MPBH), or 3-(2-pyridyldithio) propionyl hydrazide (PDPH).

The affinity binding mentioned above is preferably based on specific affinity binding to sugar residues in the core structure of the mucin molecules. The present inventors have found the carbohydrate binding proteins, collectively known as lectins, to be a suitable group of compounds for the purpose of affinity binding to the mucin matrix. Lectins having, for example, specificity towards sialic acid contained in the oligosaccharides or to specific sugar sequences in mucin can be used. Specific examples of sources of lectins and the corresponding affinity of such lectins are listed below:

| Source of lectin | Affinity |
|---|---|
| Sambucus nigra (elderberry) | towards NeuAcα2,3Gal |
| | towards NeuAcα2,6Gal |
| Limulus polyphemus (horseshoe crab) | towards NeuAcα2,6GalNAc |
| Artocarpus integrifolia (jack fruit) | towards MeαGal |
| (e.g. jacalin) | |
| Influenza virus lectin - wild type | towards NeuAc derivatives |

wherein Neu means neuraminic acid (= sialic acid).

Particles, such as cells having already existing mucin-binding surface lectins, can naturally also be bound to or captured by affinity binding to the matrix.

In some applications, such as in the case of in vivo applications where immunogenicity is an important consideration, it is preferred that the affinity molecule is not unduly large and bulky, since the immunogenicity generally can be expected to increase as a function of the size of the specific molecule. In such cases, a functionally efficient fragment of an affinity molecule is preferably used. In order to be functionally efficient, such fragment must exhibit sufficient affinity for adsorption thereof to a mucin layer to take place and also allow for linking to the specific molecule or particle of interest by methods known in the art. It is also conceivable to modify such functionally efficient fragment in order to further enhance the affinity thereof for mucin. Such modification can for example consist of the addition or altering of a few amino acid residues in one of the terminals of such fragment, especially in the mucin-binding terminal thereof.

In the case of jacalin, such functionally efficient fragment is suitably comprised of the alpha chain of the protein monomer unit containing 133 amino acids (from residues 85 to 217).

A functionally efficient fragment of the jacalin molecule can be obtained by expressing the alpha chain in BL21 E. coli strains using the pET101/D-TOPO vector. As already mentioned, such fragment can be modified in order to further enhance the affinity thereof for a mucin layer.

As an example of molecules, which can be immobilised by means of inventive method, deoxyribonucleic acid (DNA) or peptide nucleic acid (PNA) can be mentioned. DNA or PNA can be immobilised as affinity ligand on the matrix for analytical use, wherein, for example, the DNA covalently attached to the matrix is complementary to the one of interest (e.g. probing), or DNA or PNA for use in delivery applications, such as medical implants with sustained release of DNA or PNA as a biologically functional or pharmaceutically active agent. Double stranded DNA or PNA can of course also be attached to the matrix according to the invention.

In delivery applications, liposome-based ligands can also be used, wherein the liposomes are ligands to a functional molecule being immobilised on the matrix.

DNA can for example be bound to the mucin matrix by use of nick translation of DNA, i.e. by inserting a derivative with specificity towards mucin via a bifunctional linker molecule, such as a lectin rendered bifunctional, for example biotinylated jacalin. DNA can also be chemically modified by use of homobifunctional reagents such as bis-hydrazide or diamine, followed by reaction with activated mucin. Another way of binding DNA to the matrix is by immobilisation of DNA binding proteins onto the matrix. Biotin can also be incorporated into a PNA molecule for providing means for affinity binding to the matrix. As will be evident to the person skilled in the art numerous ways can be used in order to incorporate into DNA or PNA moieties having affinity for the matrix of the present invention.

Generally, macromolecules or particles can also be linked via existing biotin-avidin technology, using a bifunctional lectin such as biotinylated jacalin.

The present inventors have found that mucin, such as commercially available bovine submaxillary gland mucin, generally contains minor amounts of other, lower molecular weight proteins such as bovine serum albumin (BSA). Such molecules are believed to be present in the mucin in the form of free molecules, but are also likely to be present in the form of complexes with the mucin molecules. The complexes are primarily thought to be held together by hydrophobic and ionic interactions. The free relatively small molecules are thought to more readily diffuse from the solvent medium to a hydrophobic surface than the larger and more slowly diffusing mucin molecules. Thus, such small molecules could be adsorbed more rapidly onto a hydrophobic substrate, thereby inhibiting binding of the mucin molecules. Such impurities would then lead to a higher degree of non-specific binding to the mucin matrix. These impurities may also lead to other undesired impairment of the function of mucin in the present invention. In general, the level of low-molecular weight impurities present in the mucin used according to the present invention, which impurities are likely to compete with the mucin molecules for the binding sites of the surface of the substrate, should thus be kept low. The method of purification is not critical and can be any method or combination of methods commonly used in the art of protein purification for removing low-molecular weight fractions, such as, for example, by using ultra centrifugation, anionic exchange resins and/or gel chromatography. Accordingly, in a preferred embodiment, the method of the invention includes purification of the mucin by any conventional method or combination thereof. According to a more preferred embodiment, the method of the invention includes a step of purification of the mucin effective for disrupting complexes of mucin and lower molecular weight proteins, such as albumin, e.g. BSA, and specific removal of such lower molecular weitght molecules, for example by means of specific affinity.

The inventors have found a suitable method of purification to be one comprising the sequential use of anionic exchanger and gel filtration, gel filtration alone, or gel filtration followed by a specific affinity removal of BSA through batch adsorption by Blue Sepharose (Amersham Biosciences). By using said methods of purification, a mucin of a higher degree of purity, than the prior art mucin, is obtained. This mucin has been found to have improved properties as compared to the conventionally used commercially available prior art mucin, and thus leads to better results being obtainable according to the present invention. According to a more preferred embodiment of the method of the invention, the method includes the sequential use of anionic exchanger and gel filtration, gel filtration alone, or gel filtration followed by a specific affinity removal of BSA through batch adsorption by Blue Sepharose.

The mucin of the present invention preferably exhibits a reduced content of albumin as compared to that of conventionally available mucin, more preferably the content of albumin should be essentially absent, and most preferably, the presence of any smaller proteins or peptides in the mucin should be kept as low as possible.

### EXAMPLES

Through examples 1 and 3-11, bovine submaxillary gland mucin (BSM) (commercially available as Sigma product M3895) was used after purification by means of two alternative routes; either sequential use of anion exchanger (Q Sepharose FF from Amersham Biosciences) and gel filtration (Sepharose 6B-CL from Amersham Biosciences) or gel filtration (Sepharose 6B-CL) alone.

In Example 2 and 5, human salivary mucin (HSM) was used after purification by means of gel filtration (Sephadex G-200 in combination with Superose 6; both from Amersham Biosciences)

Although BSM and HSM were used in the examples, it is to be understood that mucin derived from other sources also can be used according to the invention, specific examples being human ocular mucin, sheep mucin, porcine mucin and the like.

### Example 1

### Purification of Bovine Submaxillary Gland Mucin (BSM)

Alternative 1. (BSM-1)

BSM (Sigma M3895) was dissolved in 20 mM piperazine containing 150 mM NaCl pH 5.0 (PipS) at 4 mg/ml. The crude sample was chromatographed on a Q Sepharose FF column (HR 10/15) pre-equilibrated with PipS buffer and adsorbed material was eluted with NaCl by gradient elution. This material was further purified on a Sepharose 6B-CL column (XK 40/26), pre-equilibrated with 50 mM ammonium acetate pH 7.0; the void fractions were pooled, lyophilized and kept desiccated at 20° C. until used.

Alternative 2. (BSM-2)

BSM (Sigma M3895) was dissolved in 25 mM ammonium acetate buffer containing 150 mM NaCl pH 7.0 at 4 mg/ml and the crude sample was chromatographed at 8° C. on a Sepharose 6B-CL column (XK 40/26) pre-equilibrated with the same buffer. The void material was further desalted against water on a Sephadex G-25 M column (Amersham Biosciences; HR 10/30), lyophilized and kept desiccated at −20° C. until used.

Alternative 3. (BSM-3)

BSM (Sigma M3895) was dissolved in 175 mM Tris buffer pH 7.4 and chromatographed on a Sephadex G-200 pre-equilibrated with the same buffer. The void material was concentrated using a Savant SpeedVac evaporator and desalted on a PD-10 column (Amersham Biosciences) against the above-mentioned Tris buffer.

### Example 2

### Purification of Human Salivary Mucin (HSM)

Human saliva was collected by expectoration without any stimulation and diluted 1:1 with 175 mM Tris buffer pH 7.4 containing 0.8 M imidazole. The diluted saliva was centrifuged at 13,000 rpm for 10 minutes and the supernatant was withdrawn, diluted twofold in running buffer, and chromatographed against 175 mM Tris buffer pH 7.4 on a Sephadex G-200 column. Pooled void material was subjected to further purification on a Superose 6 column and the void material was collected, concentrated using a Savant SpeedVac evaporator and desalted on a PD-10 column (Amersham Biosciences) before lyophilization. The purified HSM was kept desiccated at 20° C. until used.

### Example 3

### Characterisation of Mucin

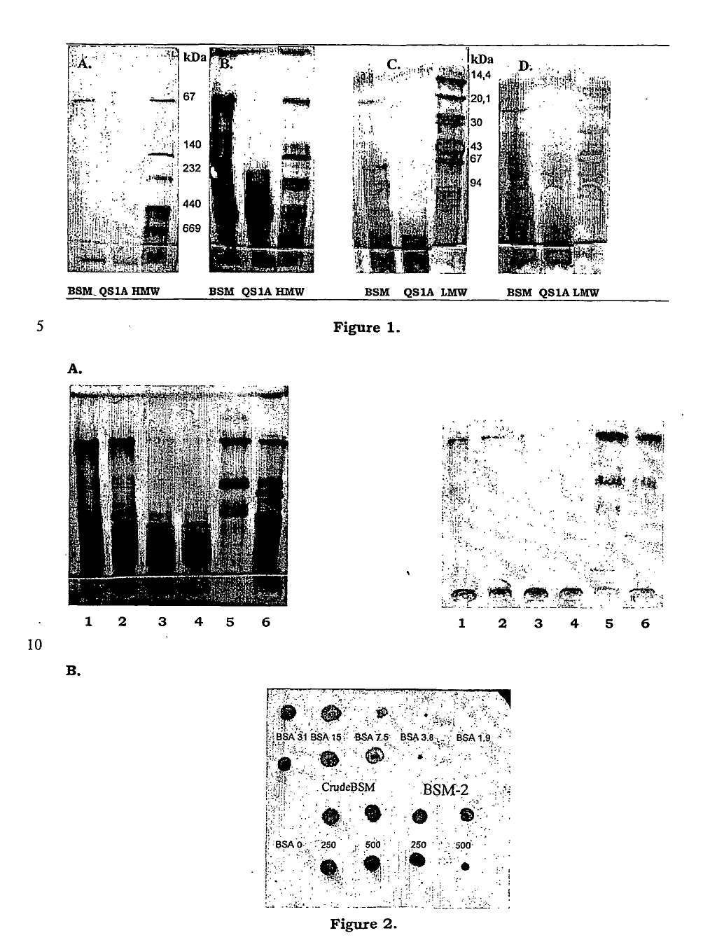

Purified mucin was analyzed by polyacrylamide gel electrophoresis (PAGE) on a Phast System using gradient 8-25 gel (Amersham Biosciences) under native and denaturing conditions and stained for proteins (Silver) and proteins/carbohydrates (combined Silver/Alcian Blue-Periodic acid protocol). FIG. 1 shows native and denaturing PAGE stained accordingly, for the purified BSM-1 fraction QS1A.

Further characterisation was performed using static light scattering, amino acid analysis and carbohydrate assay. Table 1 summarises composition of purified BSM-3 compared to reported data.

TABLE 1
Composition of purified BSM compared to reported data.

| | | Carbohydrate | Main amino acids | |
|---|---|---|---|---|
| Sample | M_{R} | content | content | Cysteine |
| Purified | 1,6 MDa | 44% | Ser/Gly/Thr/Ala/Pro | No |
| BSM¹: | | | | |
| Domain 1 | — | — | Ser/Thr/Pro | Low |
| Domain 2 | — | — | — | 11% |
| MUC7 | 150 kDa | 68% | Ser/Gly/Thr/Ala/Pro | 2 residues |
| gene | | | | |
| product¹ | | | | |

¹Gendler. S.; Spicer, A. Ann. Rev. Physiol. 1995, 57, 607

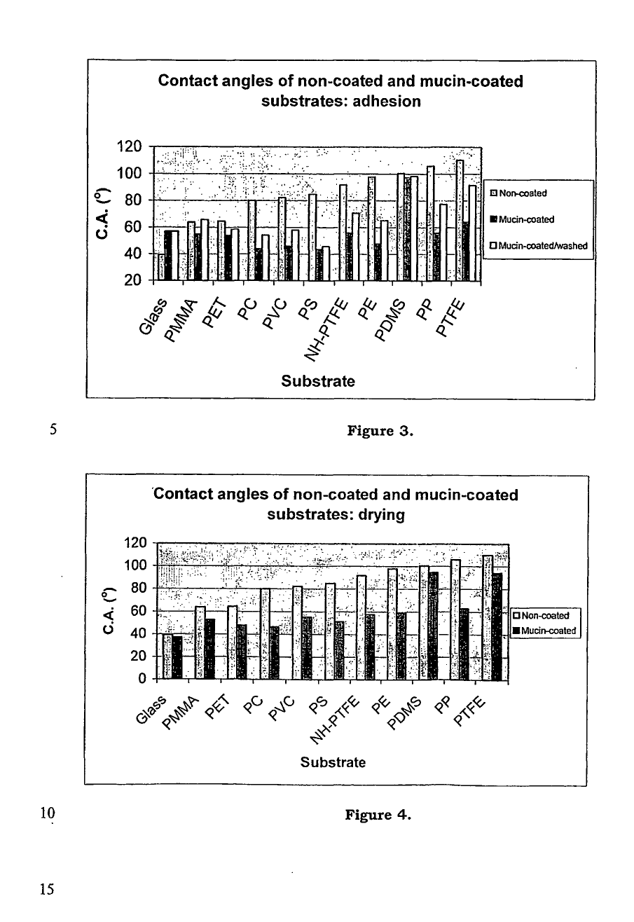

Purified BSM-2 was specifically screened for BSA by Western blot of PAGE gels and by whole sample analysis on nitrocellulose membrane using an anti-BSA antibody amplified by the alkaline phosphatase/BCIP-NBP system system (all components from Sigma). FIGS. 2 and 3 show the results of these experiments.

BSA estimates based on integral analysis (BioRad Molecular Analysis version 1.3) of combined Silver/Alcian Blue-Periodic acid stained gels indicate that purified BSM-2 contains approximately 0.7% BSA. That figure reaches approximately 6.4% in the case of crude BSM. Western blot shows that BSA is also found in the high molecular weight portion of the mucin preparation, something leading to a somewhat higher total BSA content.

Whole sample volume analysis (BioRad Molecular Analysis version 1.3) with BSA as standard estimates the BSA content to be approximately 2.3%.

### Example 4

### Coating of Hydrophobic Surfaces: Contact Angle Measurements

Substrates, approximately 1 cm² in size, of polystyrene (PS), polypropylene (PP), polyethylene (PE), polyvinylchloride (PVC), polydimethyl siloxane (PDMS, also referred to as MQ), polybisphenol A carbonate (PBAC), polyethylene terephtalate (PET), polymethyl methacrylate (PMMA), polytetrafluoroethylene (PTFE) and aminated polytetrafluoroethylene (NH-PTFE) respectively were rubbed and washed with isopropanol before sonication for 10 minutes in the same solvent. The sonciated substrates were then thoroughly rinsed and dried with nitrogen gas.

Washed polymers were coated with BSM-2 at 0.5 mg/ml in TBS pH 7.4 over night at 37° C. and washed according to following: immersion 5 times in TBS before rinsing in MilliQ water and drying with nitrogen.

FIG. 3 shows the results of water contact angle measurements of polymers before and after coating with purified BSM-2. Contact angles were also measured after controlled TBS wash of mucin-coated substrate for 1 hour at shear rate 50 s⁻¹. Glass served as a hydrophilic control. It can be seen that mucin-coated substrates show significantly lower contact angles as compared to non-coated controls except for the case of silicone and glass control substrate. These data indicate that a layer of mucin has adhered to the surface, hence giving it a more hydrophilic character. In addition, the coatings withstand wash at high shear rate for 1 hour.

Alternatively, the mucin coating is dried onto the substrates by incubation at 37° C. FIG. 4 shows contact angle data obtained by this coating technique. It can be seen that contact angles are reduced similar to the case of adhesion by dip-coating.

### Example 5

### Lowered Non-Specific Binding of Proteins

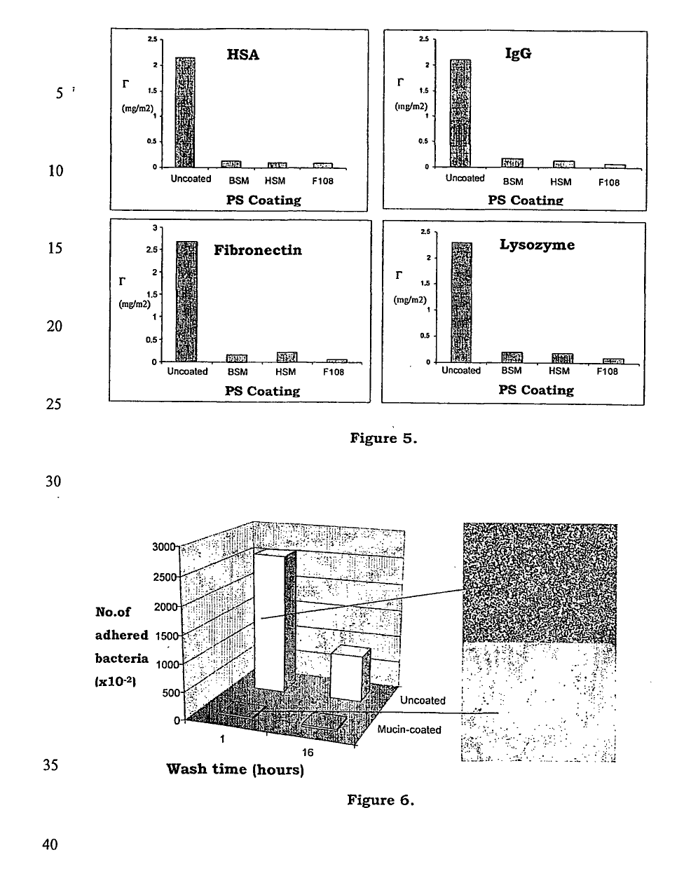

Polystyrene (PS) particles with a diameter of 282 nm were pre-coated with purified BSM-3 or purified HSM at approximately 0.5 mg/ml in Phosphate buffer containing 150 mM NaCl (PBS) for 24 hours at room temperature. Pluronic F108 and bare PS particles served as controls. Pre-coated particles were washed twice in TBS by means of repeated centrifugation/wash cycles and the four model proteins, human serum albumin (HSA), human plasma fibronectin, immunoglobulin G (IgG) and lysozyme, were incubated at 0.5 mg/ml for 24 hours at room temperature. The particles were, after washing twice in PBS and drying using a SpeedVac evaporator (Savant), subjected to amino acid analysis and the protein uptake is summarized in FIG. 5.

It should be noticed that in the case of the BSM-3- and HSM-coated particles, a residual amount of protein originates from the coating itself.

### Example 6

### Suppression of the Bacteria Pseudomonas aeruginosa

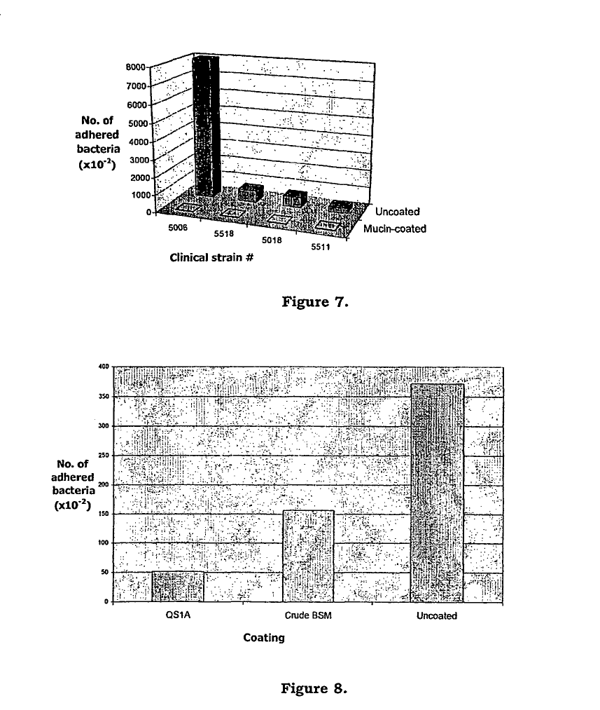

Polystyrene surfaces were mucin-coated with BSM-1 by drying according to Example 4 and washed with TBS pH 7.4 in a flow-cell for different times at shear rate 29 s⁻¹. The washed substrates were thereafter incubated for 30 minutes with different strains of the human pathogen Pseudomonas aeruginosa. The numbers of adhered bacteria are presented in FIGS. 6 and 7.

Furthermore the bacterial suppressing capacity was compared between crude and purified BSM after 30 minutes wash. The results are shown in FIG. 8.

The mucin matrix of the invention has unexpectedly been found to suppress colonisation by Pseudomonas aeruginosa, which is commonly known to specifically bind to mucous surfaces.

### Example 7

### Reduced Cell Adhesion

Polystyrene microtiter wells were coated over night at room temperature with purified BSM-1 at 1 mg/ml in PBS pH 7.4. After washing 3 times in PBS, wells were incubated with human fibronectin, human hyaluronic acid and BSA respectively at 0.5 mg/ml for 1 hour at room temperature. Human osteoblast cell line MG63 was, after additonal wash steps, seeded and grown for 24 hours at 37° C. in CO₂ atmosphere. PBS served as control. Relative cell count is summarised in Table X.

TABLE 2
Relative cell count for mucin-coated polystyrene surfaces
treated with BSA, hyaluronic acid and fibronectin respectively.

| Secondary coating | Mucin-coated | Non-coated (PBS) |
|---|---|---|
| PBS | − | + |
| Bovine Serum Albumin | − | + |
| Hyaluronic acid | − | ++ |
| Fibronectin | − | ++ |
| Bare PS (control) | ND | +− |

PBS served as control.
Symbols:
++ = many cells;
+ = cells;
+− = cells and dead cells;
− = no cells.

From the above table it can clearly be seen that mucin-coated surfaces reduce cell adhesion compared to non-coated controls. Furthermore, in conjunction with Example 5, it can be seen that specific binding of cell-stimulating proteins is reduced.

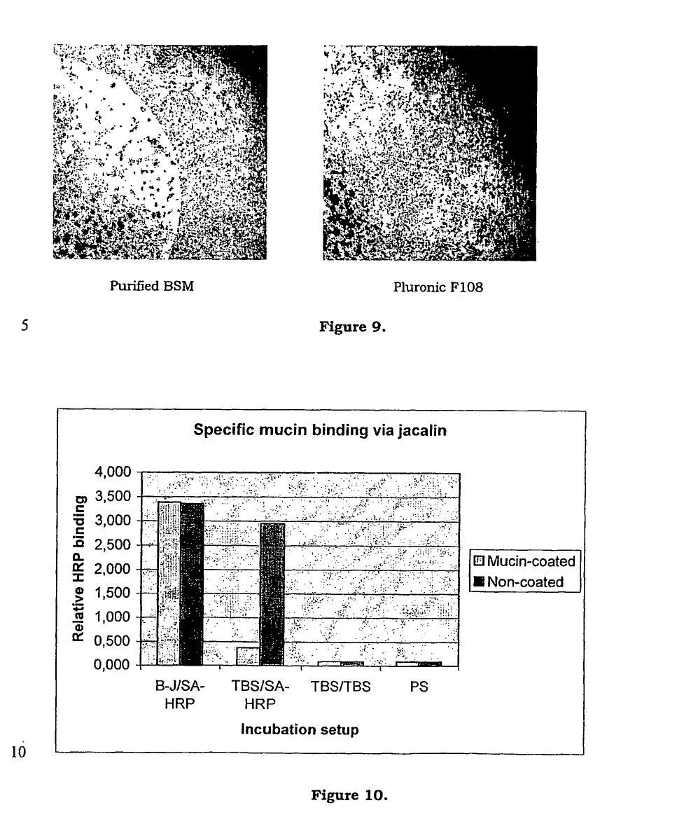

In another experiment setup, human peripheral neuronal blastoma cells (SH 45 Y) were seeded on polystyrene surfaces which had been pre-treated by drop-coating with purified BSM-1 at 0.25 mg/ml and Pluronic F108 at 1 mg/ml, respectively, and subsequent coateding with human fibronection at 0.5 mg/ml. FIG. 9 shows light microscopy pictures of drop interface area.

### Example 8

### Specific Binding Via the Jacalin System

Polystyrene microtiter wells were mucin-coated (BSM-2) over night at 37° C. and after wash 3 times with TBS sequentially incubated with biotinylated jacalin (B-J) at 250 μg/ml (Pierce) and streptavidin-horse radish peroxidase conjugate (SA-HRP) at 10 μg/ml (Vector Laboratories). All incubations were performed for 1 hour with shaking at room temperature. Surface-associated HRP were estimated, after final wash in TBS, by development with ABTS substrate (Boehringer Mannheim GmbH) for 3 minutes and absorbance measured at 405 nm. Non-coated polystyrene served as control surface and TBS together with B-J and SA-HRP as control components. FIG. 10 shows the relative HRP binding (absorbance value at 405 nm) for each and every incubation setup.

### Example 9

### Mucin Chemically Modified and Covalently Bound to Aminated PTFE

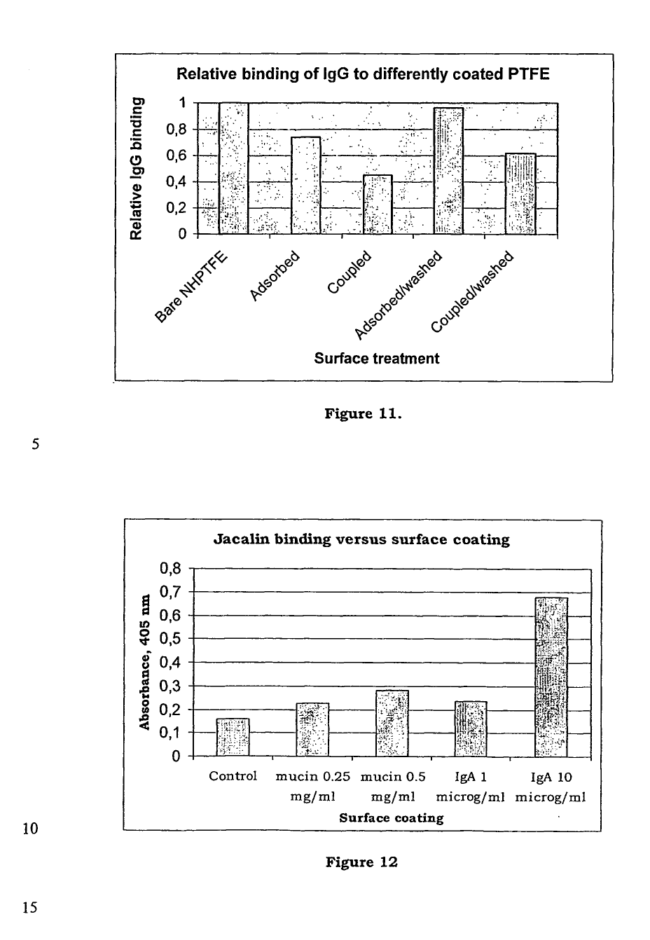

Polytetrafluoroethylene (PTFE) foil of 0.2 mm was amine-functionalized by plasma-treatment according to the following: first the polymeric material is pretreated with O₂ at 8 ml/min and 14 MHz/100 W for 30 seconds, and thereafter the substrate is aminated with diaminocyclohexane (DACH), at 18 mTorr and 170 kHz/10 W for 2 minutes. Aminated PTFE (NH-PTFE) was stored at 8° C. and high humidity. Purified BSM-1 at 2 mg/ml was periodate-activated by reaction on ice for 30 minutes with 1 mM sodium periodate in PBS pH 7.0. Activated BSM-1 was thereafter gel filtrated on a PD-10 column against PBS pH 8.0 and incubated with aminated PTFE for 6 hours at 8° C. After washing two times in PBS pH 8.0 the resulting Shiff's base was reduced with 5 mM ascorbic acid in PBS pH 8.0 for 1 hour at room temperature. After additional wash and blocking with 1% ovalbumin, the chemically modified, BSM-coupled, NH-PTFE surface was tested for lowered non-specific binding using an IgG antibody system amplified by the alkaline phosphatase/BCIP-NBP system. Stability of chemically coupled versus adsorbed BSM-1 was further tested by sonication in 1 M NaCl/1.25% sodium dodecylsulfate (SDS) for 30 seconds before antibody visualization. FIG. 11 summarises relative IgG binding compared to non-treated NH-PTFE.

### Example 10

### Production of Minimised Jacalin and Demonstration of Its Binding to Mucin

Minimised jacalin consisting of a methionine-terminated (5′ end) 133 amino acid sequence, with carbohydrate-binding ability (alpha chain; residue 85 to 217), was constructed by PCR amplification of target sequence from native jacalin. The amino acid sequence for the constructed alpha chain region of native jacalin is the following:

MGKAFDDGAFTGIREINLSYNKETAIGDFQVVYDLNGSPYVGQNHKSFIT
GFTPVKISLDFPSEYIMEVSGYTGNVSGYVVVRSLTFKTNKKTYGPYGVT
NGTPFNLPIENGLIVGFKGSIGYWLDYFSMYLSL

PCR amplification was performed using the following primers:

| | |
|---|---|
| Forward | 5′ CACCATGGGTAAAGCTTTTGATGACGGTG 3′ |
| Reverse | 5′ AAGTGACAAGTACATACTAAAGTAG 3′ |

The construct was subjected to sequencing and was shown to match cDNA clone pSKcJA17 presented by Hui Yang and Thomas H. Czapla (JBC 1993, volume 268, pp. 5905-5910) with one single amino acid replacement at residue 184 (serine replaced by asparagine).

The amplified alpha chain sequence was inserted into a pET101/D-TOPO vector system (polyhistidine frame) and expressed using BL21 E. coli strains. Bacteria were cultivated at 37° C. in LB medium containing 50 μg ampicillin/ml and protein expression was induced using 1 mM IPTG.

The expressed minimised jacalin was further subjected to binding experiments using purified BSM: polystyrene microtiter wells were coated at 0.25 and 0.5 mg/ml with purified BSM in PBS pH 7.4 at room temperature over night. Human immunoglobulin A (IgA) was used as a positive control at 1 and 10 μg/ml diluted in 50 mM sodium hydrogencarbonate pH 9.6.

After coating with BSM and subsequent block with 1% BSA in PBS (blocking buffer) for 1 hour at 37° C., wells were incubated with expressed jacalin alpha chain fragment for 3 hours at 37° C. Thereafter the wells were washed 3 times with PBS pH 7.4. The next-following step includes incubation with anti-histidine (C-terminal)-horse radish peroxidase conjugate (a-His-HRP; Invitrogen) diluted 1:800 in blocking buffer for 1.5 hours at 37° C. After additional washing the wells were developed using ABTS substrate (Boehringer Mannheim GmbH). Developing time was 40 minutes at 37° C. and absorbance measurements were performed at 405 nm. The results are shown in FIG. 12.

### Example 11

### Biocompatibility Compared to Fibrinogen and Pluronic F108

The tolerance for implanted polyurethane (PU) model surfaces with different coatings was tested in a preliminary screen using a sheep model. After 30 days the test surfaces, which had been joined together during the experiment, were removed so that the neighbouring tissues could be subjected to histological evaluation. This cursory comparison indicated an excitingly low level of inflammatory cells, and minimal capsule formation, in those tissues that had been in contact with surfaces coated with the high molecular weight BSM mucin fraction (BSM-3), compared to those in contact with Fibrinogen and Pluronic F108.

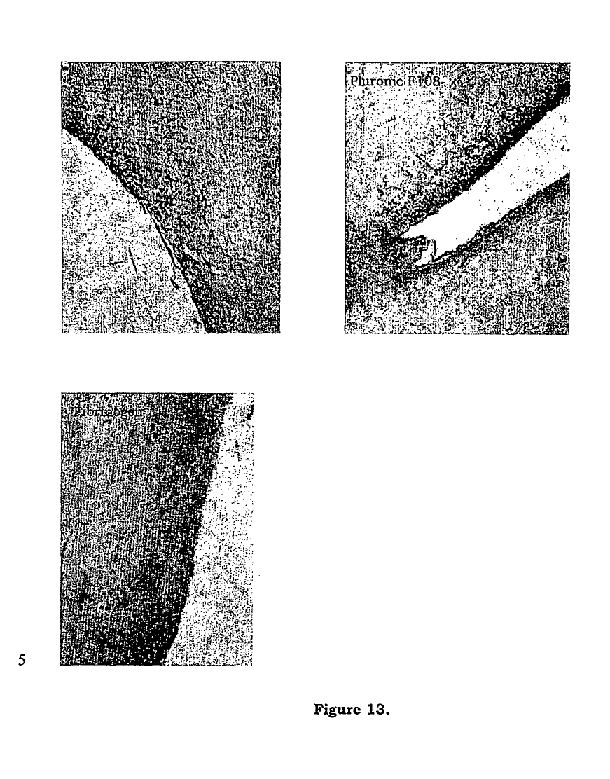

FIG. 13 shows implant surfaces after removal and staining as known in the art. It can be seen in the case of mucin-coated PU that the capsule consists of dense and well-vascularized collagen. Furthermore the inner margin of the formed 30 μm capsule is well organized suggesting that the encapsulation phase was resolving. Compared to fibrinogen and Pluronic F108 coated PU the mucin-coated PU implant has noticeably less inflammatory infiltrates. In addition, there is evidence of neovascularity in the surrounding of the mucin-coated implant.

1. A method of immobilising selected molecules and/or particles to a surface of a hydrophobic polymeric material, characterised in comprising the following steps:
forming a mucin layer onto a surface of a substrate of a hydrophobic polymeric material; and
adsorbing and/or covalently attaching said molecules and/or particles to the mucin layer formed.

2. The method of claim 1, characterised in that the polymeric material is a biomaterial.

3. The method of claim 1 or 2, characterised in that the mucin layer is formed by adsorption to said hydrophobic surface.

4. The method of any of the previous claims, characterised in comprising chemical activation of an exposed functional group of the mucin molecules of the mucin layer.

5. The method of claim 4, characterised in that the activation is accomplished by mild oxidation.

6. The method of any of the previous claims, characterised in adsorbing molecules and/or particles having affinity for the mucin.

7. The method of any of the previous claims, characterised in comprising linking of any of said molecules and/or particles to a molecule or functional fragment thereof having affinity for the mucin.

8. The method of claim 7, characterised in that the molecule or functional fragment thereof is a lectin molecule or an, optionally modified, functional fragment thereof, and more preferably jacalin or a functional fragment thereof.

9. The method of claim 8, characterised in that the functional fragment comprises the sequence of the amino acid residues number 85 to 217 of jacalin.

10. The method of any of the previous claims, characterised in further comprising a purification step effective for reducing the level of free albumin in the mucin.

11. The method of claim 10, characterised in that the purification step comprises the sequential use of anionic exchanger and gel filtration, gel filtration alone, or gel filtration followed by a specific affinity removal of free albumin through batch adsorption, or a combination thereof.

12. A hydrophobic polymeric substrate provided with a mucin layer having selected molecules and/or particles immobilised thereto, obtainable by means of the method of any of claims 1-11.

13. The hydrophobic polymeric substrate of claim 12, characterised in comprising a pharmaceutical article for implantation in the human body.

14. A biocompatible highly specific binding system comprising a mucin and a lectin or a functional fragment of a lectin, which system can be used in the method of claim 1.

15. The biocompatible highly specific binding system according to claim 11, wherein the lectin is jacalin or a functional fragment thereof.


SEQUENCE LISTING (3 sequences)

SEQ ID NO: 1 - protein, 134 aa, Artificial Sequence
  Methionine-terminated (5′ end) amino acid sequence for the alpha chain region of native jacalin
MGKAFDDGAFTGIREINLSYNKETAIGDFQVVYDLNGSPYVGQNHKSFITGFTPVKISLDFPSEYIMEVSGYTGNVSGYVVVRSLTFKTNKKTYGPYGVTNGTPFNLPIENGLIVGFKGSIGYWLDYFSMYLSL

SEQ ID NO: 2 - DNA, 29 bp, Artificial Sequence
  Synthetic Oligonucleotide
gtggcagtagttttcgaaatgggtaccac

SEQ ID NO: 3 - DNA, 25 bp, Artificial Sequence
  Synthetic Oligonucleotide
gatgaaatcatacatgaacagtgaa